(19) United States
(12) Patent Application Publication
(10) Pub. No.: US 2013/0089519 A1
(43) Pub. Date: Apr. 11, 2013

(54) Method for Predicting a Therapy Response in Subjects with Multiple Sclerosis

(71) Applicants: Richard A. Rudick, Solon, OH (US); Richard M. Ransohoff, Shaker Heights, OH (US)

(72) Inventors: Richard A. Rudick, Solon, OH (US); Richard M. Ransohoff, Shaker Heights, OH (US)

(21) Appl. No.: 13/704,752

(22) PCT Filed: Jun. 17, 2011
(86) PCT No.: PCT/US11/40810

§ 371 Date: Dec. 17, 2012

Related U.S. Application Data
(60) Provisional application No. 61/356,265, filed on Jun. 18, 2010.

(51) Int. Cl. C12Q 1/68

(52) U.S. Cl. CPC C12Q 1/6837
USPC 424/85.6; 506/9

(57) ABSTRACT

A method is provided for determining the efficacy of interferon-beta (IFN-β) therapy in a subject with multiple sclerosis. One step of the method can include obtaining a biological sample from the subject. After obtaining the biological sample, the expression level of at least one interferon-regulated gene (IRG) and/or variant thereof can be determined. Increased or decreased expression of the at least one IRG and/or variant thereof as compared to a control may indicate that the subject will respond poorly to IFN-β therapy.

### RELATED APPLICATION

This application claims priority from U.S. Provisional Patent Application Ser. No. 61/356,265, filed Jun. 18, 2010, the entirety of which is hereby incorporated by reference.

### TECHNICAL FIELD

The present invention generally relates to methods for predicting a therapy response in subjects with multiple sclerosis (MS), and more particularly to a method for predicting a response to IFN-β therapy in subjects with MS based on differentially expressed genetic markers.

### BACKGROUND OF THE INVENTION

Multiple sclerosis (MS) is an inflammatory disease of the central nervous system. Genome-wide association studies have implicated immune system genes in MS disease susceptibility, which is consistent with a role for immune mechanisms in MS pathogenesis. Increased bioavailability of type I interferon (IFN) has been implicated in susceptibility or severity of diverse autoimmune disorders. Increased expression of type I IFN-regulated genes (IRGs) has been detected in about 50% of untreated MS patients, and this has been interpreted as delineating a subset of patients with augmented innate immunity.

Types I and II IFNs regulate overlapping sets of IRGs. While type I IFN is a cardinal mediator of innate immunity, type II IFN participates in both innate and adaptive immunity. Although clinical trials for IFN-γ as a therapeutic agent for MS were unsuccessful, clinical trials of type I IFN continued and several recombinant interferon-beta (IFN-β) products have been approved for MS. In the trials, IFN-β reduced relapse rates by 30% and inhibited brain lesion formation visualized by magnetic resonance imaging. Clinical responses varied among individuals, however, and the mechanism(s) of action remained obscure.

In post-hoc data analyses from one of the phase 3 trials, about 20% of IFN-β recipients were identified as poor responders (PR). Poor response status has recently been categorized as pharmacologic (i.e., related to production of IFN-β neutralizing antibodies) or pharmacogenomic (i.e., associated with genetic variants in IFN-β receptors or signalling components). These patients share in common reduced IFN-β bioavailability. Despite this mechanistic clarity, such patients account for a minority of PRs. In the third and largest category, PR to IFN-β may be related to the nature of the IFN-β response, which may be informative regarding the pathogenesis of MS in a subset of patients. Microarray-based cross-sectional expression analyses and studies of individual candidate genes support this concept.

All these clinical and radiological variables, however, are limited in their ability to predict disease outcome, especially during early stages of MS. This uncertainty in forecasting disease outcome means that some MS patients who need aggressive treatment do not receive it, while others are unnecessarily treated and as a result are exposed to the risk of side effects without a sound rationale.

### SUMMARY OF THE INVENTION

The present invention generally relates to methods for predicting a therapy response in subjects with multiple sclerosis (MS), and more particularly to a method for predicting a response to interferon-beta (IFN-β) therapy in subjects with MS based on differentially expressed genetic markers. According to one aspect of the present invention, a method is provided for determining the efficacy of IFN-β therapy in a subject with MS. One step of the method can include obtaining a biological sample from the subject. After obtaining the biological sample, the expression level of at least one interferon-regulated gene (IRG) or variant thereof can be determined. Increased or decreased expression of the at least one IRG or variant thereof as compared to a control may indicate that the subject will respond poorly to IFN-β therapy.

According to another aspect of the present invention, a method is provided for screening an agent that can be used to treat MS. One step of the method can include providing a population of peripheral blood mononuclear cells (PBMCs) from a subject with MS that is a poor responder to IFN-β therapy. Next, an agent can be administered to the PBMCs. The expression level of at least one IRG or variant thereof can then be determined in one or more of the PBMCs.

According to another aspect of the present invention, a method is provided for treating a subject with MS. One step of the method can include obtaining a biological sample from the subject. After obtaining the biological sample, the expression level of at least one IRG or variant thereof can be determined. If expression of one or more of the at least one IRG or variant thereof is increased or decreased as compared to a control, the subject can be administered a therapeutically effective amount of at least one agent besides IFN-β.

According to another aspect of the present invention, a method is provided for treating a subject with MS. One step of the method can include obtaining a biological sample from the subject. After obtaining the biological sample, the expression level of at least one ERG or variant thereof can be determined. If expression of the at least one IRG or variant thereof is increased or decreased as compared to a control, the subject can be administered a therapeutically effective amount of natalizumab.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a flow diagram illustrating a method for determining the efficacy of interferon-beta (IFN-β) therapy in a subject with multiple sclerosis (MS) according to one aspect of the present invention;

FIG. 2 is a flow diagram illustrating a method for screening an agent that can be used to treat MS according to another aspect of the present invention;

FIG. 3 is a flow diagram illustrating a method for treating a subject with MS according to another aspect of the present invention;

FIG. 4 is a scatter plot showing the correlation between induction ratios (IRs) for OASL calculated by real-time quantitative PCR vs macroarray (a log 2 scale is shown for the X and Y axes);

FIG. 5 is a plot showing the number of interferon-regulated genes (IRGs) at first IFN-β injection. The bars represent individual subjects at the initial IFN-β injection. The height of the bars shows the number of IRGs with IRs ≧2.0. The patients with poor treatment response are shaded;

FIG. 6 shows a series of scatter plots for 85 patients for the IFN-β molecular response at baseline (x-axis) and 6-months (y-axis). For each subject, the IR for each of 166 genes is shown at the two time points. Variability of the molecular response between the two time points is indicated by deviation from the diagonal line in each plot;

FIG. 7 is a series of scatter plots for 10 individual patients showing consistent response over 24 months. Ten patients with MS (5 good and 5 poor responders) with macroarray data at baseline, 6 months, and 24 months were randomly selected to test the consistency of the response over 2 years. The first 3 columns are patients with poor treatment response, and the last 3 columns are patients with good treatment response. Columns 1 and 4 compare responses at baseline and 6 months. Columns 2 and 5 compare responses at 6 and 24 months. Columns 3 and 6 compare responses at baseline and 24 months;

FIGS. 8A-B are a series of histograms showing exaggerated IRG response in patients with a poor response at first IFN-β injection (FIG. 8A) and a 6-month IFN-β injection (FIG. 8B) (histograms plot the IR for all genes in all patients in the good response group and all patients in the poor response group); and

FIG. 9 is a plot showing ROC curves for baseline T2 lesion volume (LV), the best 25 IRGs at baseline, and baseline T2 lesion volume+the best 25 IRGs. The ROC curve tests the ability of 25 IRGs, measured at baseline, to predict poor response measured 6-months later, and compares the predictive ability with the baseline T2 lesion volume.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the present invention.

In the context of the present invention, the terms “control” or “control sample” can refer to any subject sample or isolated sample that serves as a reference.

As used herein, the term “mRNA” can refer to transcripts of a gene. Transcripts can include RNA, such as mature mRNA that is ready for translation and/or at various stages of transcript processing (e.g., splicing and degradation).

As used herein, the terms “nucleic acid” or “nucleic acid molecule” can refer to a deoxyribonucleotide or ribonucleotide chain in either single- or double-stranded form, and can encompass known analogs of natural nucleotides that function in a similar manner as naturally occurring nucleotides.

As used herein, the terms “polypeptide” and “protein” can refer to a molecule that comprises more than one amino acid subunit. A polypeptide may be an entire protein or it may be a fragment of a protein, such as an oligopeptide or an oligopeptide. The polypeptide may also comprise alterations to the amino acid subunits, such as methylation or acetylation.

As used herein, the term “probe” can refer to an oligonucleotide capable of binding to a target nucleic acid of complementary sequence through one or more types of chemical bonds, usually through complementary base pairing. For example, an oligonucleotide probe may include natural (i.e., A, G, C or T) or modified bases (e.g., 7-deazaguanosine, inosine, etc.). In addition, the bases in an oligonucleotide probe may be joined by a linkage other than a phosphodiester bond, so long as it does not interfere with hybridization.

As used herein, the term “quantifying” when used in the context of quantifying transcription levels of a gene can refer to absolute or relative quantification. Absolute quantification may be accomplished by inclusion of known concentration(s) of one or more target nucleic acids (e.g., control nucleic acids) and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, transcription level.

As used herein, the term “relative gene expression” or “relative expression” in reference to a gene can refer to the relative abundance of the same gene expression product, usually an mRNA, in different cells or tissue types.

As used herein, the term “subject” can refer to any animal, including, but not limited to, humans and non-human animals (e.g., rodents, arthropods, insects, fish), non-human primates, ovines, bovines, ruminants, lagomorphs, porcines, caprines, equines, canines, felines, ayes, etc.), which is to be the recipient of a particular diagnostic and/or therapeutic application.

As used herein, the term “biological sample” can refer to a bodily sample obtained from a subject or from components thereof. For example, the bodily sample can include a “clinical sample”, i.e., a sample derived from a subject. Such samples can include, but are not limited to: peripheral bodily fluids, which may or may not contain cells, e.g., blood, urine, plasma, mucous, bile pancreatic juice, supernatant fluid, and serum; tissue or fine needle biopsy samples; and archival samples with known diagnosis, treatment, and/or outcome history. Bodily samples may also include sections of tissues, such as frozen sections taken from histological purposes. The term “biological sample” can also encompass any material derived by processing a bodily sample. Derived materials can include, but are not limited to, cells (or their progeny) isolated from the biological sample, proteins, and/or nucleic acid molecules extracted from the sample. Processing of the biological sample may involve one or more of filtration, distillation, extraction, concentration, fixation, inactivation of interfering components, addition of reagents, and the like.

As used herein, the terms “interferon-regulated gene” or “IRG” can refer to any gene or variant thereof whose expression is increased or decreased relative to a control upon exposure to at least one interferon, such as IFN-β. Examples of IRGs can include those listed in Table 1, as well as others that are known in the art (see, e.g., Samarajiwa, S. A. et al., Nucleic Acids Res. 37:D852-D857, January 2009).

As used herein, the term “variant” when used with reference to an IRG can refer to any alteration in the IRG nucleotide sequence, and includes variations that occur in coding and non-coding regions, including exons, introns, and untranslated sequences. Variations can include single nucleotide substitutions, deletions of one or more nucleotides, and insertions of one or more nucleotides. Examples of IRG variants are known in the art (see, e.g., Vosslamber, S. et al., “Interferon regulatory factor 5 gene variants and pharmacological and clinical outcome of Interferonβ therapy in multiple sclerosis”, Genes and Immunity, published online Apr. 7, 2011; and Baranzini et al., Hum. Mol. Genet. 15:767, 2009).

The present invention generally relates to methods for predicting a therapy response in subjects with multiple sclerosis (MS), and more particularly to a method for predicting a response to interferon-beta (IFN-β) therapy in subjects with MS based on differentially expressed genetic markers. The present invention is based on the discovery that expression of interferon-regulated genes (IRGs) differs qualitatively (i.e., identity of regulated IRGs) and quantitatively (i.e., numbers of regulated IRGs and extent of induction or repression) in a subset of subjects with MS. In particular, it was unexpectedly discovered that subjects with MS who were classified as poor responders showed a significant exaggerated molecular response (i.e. increased and decreased gene expression) following first and 6-month IFN-β injections. Based on this discovery, the present invention provides a method for determining the efficacy of IFN-β therapy in a subject with MS, a method of determining whether a subject with MS should be treated with a therapeutic agent other than IFN-β, a method for screening an agent that can be used to treat MS, and methods for treating a subject with MS.

Mechanistic proposals for MS pathogenesis have focused on adaptive immunity, particularly immune response directed against myelin constituents. As noted above, it has been unexpectedly discovered that IFN-β recipients who were destined for poor responder status already had higher levels of disease activity and disease burden. Without wishing to be bound by theory, it is believed that an augmented response to type I IFN accompanies innate-immune processes that drive autoimmune pathogenesis in a subset of subjects (i.e., poor responders) with MS. Thus, it is believed that differences in innate immunity, either within type I IFN pathways or affecting the expression levels of IRGs indirectly, are determinants for enhanced disease severity in poor responders.

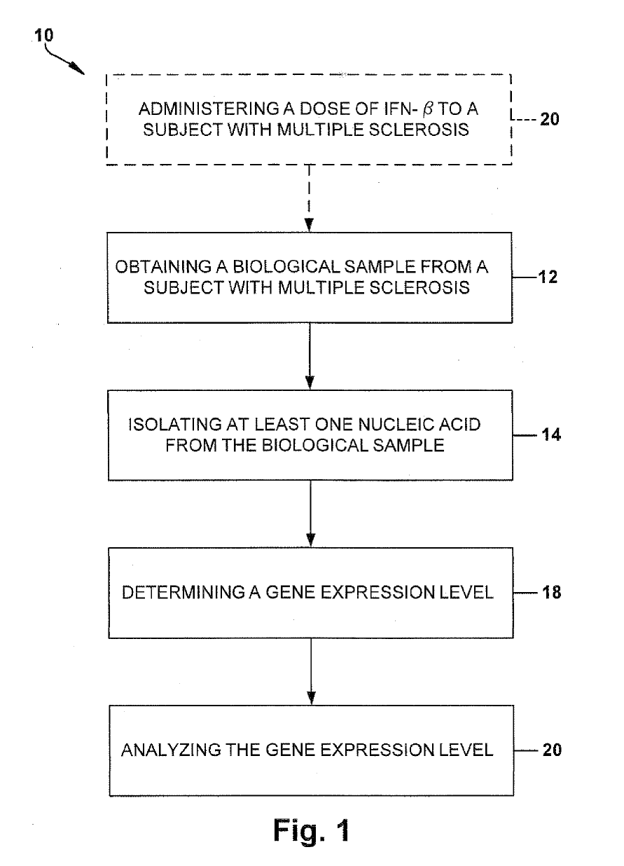

FIG. 1 is a flow diagram illustrating a method 10 in accordance with one aspect of the present invention for determining the efficacy of IFN-β therapy in a subject with MS. The method 10 can include the steps of: obtaining a biological sample from a subject with MS (Step 12); isolating at least one nucleic acid from the biological sample (Step 14); determining the expression level of at least one IRG and/or variant thereof (Step 16); and analyzing the measured gene expression level to determine if the subject will respond poorly to IFN-β therapy (Step 18). Optionally, the method 10 can include administering a dose of IFN-β to a subject with MS prior to obtaining the biological sample (Step 20).

The terms “multiple sclerosis” or “MS” as used herein can include a disease in which the fatty myelin sheaths around the axons of the brain and spinal cord are damaged, leading to demyelination and scarring. MS can include a number of subtypes, any one of which a subject may be afflicted with. Examples of MS subtypes can include benign MS, quiescent relapsing-remitting MS, active relapsing-remitting MS, primary progressive MS, and secondary progressive MS. Relapsing-remitting MS can include a clinical course of MS that is characterized by clearly defined, acute attacks with full or partial recovery and no disease progression between attacks. Primary progressive MS can include a clinical course of MS that presents initially in the progressive form with no remissions. Secondary progressive MS can include a clinical course of MS that is initially relapsing-remitting, and then becomes progressive at a variable rate, possibly with an occasional relapse and minor remission. Progressive relapsing MS can include a clinical course of MS that is progressive from the onset, punctuated by relapses. Typically, there is significant recovery immediately following a relapse, but between relapses there can be a gradual worsening of disease progression.

Referring to FIG. 1, at least one biological sample can be obtained from a subject with MS at Step 12. The term “biological sample” is used herein in its broadest sense and can include any clinical sample derived from the subject. Examples of biological samples can include, but are not limited to, peripheral bodily fluids, tissue or fine needle biopsy samples, and archival samples with known diagnosis, treatment and/or outcome history. Biological samples may also include sections of tissues, such as frozen sections taken from histological purposes, as well as any material(s) derived by processing the sample. In one example of the present invention, the biological sample can include a whole blood sample obtained using a syringe needle from a vein of a subject with MS.

At Step 14, at least one nucleic acid can be isolated from the biological sample. Nucleic acids can be isolated from the biological sample according to any of a number of known methods. One of skill in the art will appreciate that where alterations in the copy number of a gene are to be detected, genomic DNA can be isolated. Conversely, where detection of gene expression levels is desired, RNA (i.e., mRNA) can be isolated. Methods of isolating nucleic acids, such as mRNA are well known to those of skill in the art. (See, e.g., Chapter 3 of Laboratory Techniques in Biochemistry and Molecular Biology: Hybridization With Nucleic Acid Probes, Part I. Theory of Nucleic Acid Preparation. P. Tijssen, ed. Elsevier, N.Y. (1993)).

In one example of the present invention, RNA can be isolated ex vivo from a whole blood sample using a commercially available kit, such as the PAXGENE RNA blood extraction kit (PREANALYTIX, Switzerland). Briefly, at least one whole blood sample can be obtained from a subject with MS and then collected in a test tube (e.g., an RNase-free tube). Purification can begin with a centrifugation step to pellet cells in the tube. The pellet can then be washed, resuspended, and incubated in optimized buffers (together with proteinase K) to promote protein digestion. An additional centrifugation step can be carried out to homogenize the cell lysate and remove residual cell debris. Next, the supernatant of the flow-through fraction can be transferred to a fresh microcentrifuge tube. Ethanol can then be added to adjust binding conditions, followed by application of the lysate to a spin column. During a brief centrifugation, RNA can selectively bind to the membrane of the spin column as contaminants pass through. Remaining contaminants can then be removed in several efficient wash steps. Between the first and second wash steps, for example, the membrane may be treated with DNase I to remove trace amounts of bound DNA. After the wash steps, RNA may be eluted in elution buffer and heat-denatured. RNA quality and quantity can then be assessed (e.g., by spectroscopy) with additional visualization by agarose gel electrophoresis.

At Step 16, the expression level of at least one IRG and/or variant thereof can be determined from the nucleic acid(s) isolated from the biological sample. In one example of the present invention, the expression level of at least one IRG and/or variant thereof (e.g., about 4 IRGs and/or variants thereof) listed in Table 1 can be determined from the nucleic acid(s) isolated from the biological sample. In another example of the present invention, the expression level of at least one IRG and/or variant thereof (e.g., about 4 IRGs and/or variants thereof) listed in Table 3 can be determined from the nucleic acid(s) isolated from the biological sample. One of skill in the art will appreciate that to measure the expression level (and thereby the transcription level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript can include a nucleic acid for whose synthesis the mRNA transcript (or a subsequence thereof) has ultimately served as a template. Thus, a cDNA reverse transcribed from an mRNA, an RNA transcribed from that cDNA, a DNA amplified from the cDNA, an RNA transcribed from the amplified DNA, etc., can be derived from the mRNA transcript and detection of such derived products may be indicative of the presence and/or abundance of the original transcript in the sample.

Methods for detecting gene expression levels and/or activity are known in the art. Non-limiting examples of methods for detecting RNA, for example, can include Northern blot analysis, RT-PCR, RNA in situ hydridization (e.g., using DNA or RNA probes to hybridize RNA molecules present in a sample), in situ RT-PCR, and oligonucleotide microarrays (e.g., by hybridization of polynucleotide sequences derived from a sample to oligonucleotides attached to a substrate).

In one example of the present invention, a macroarray can be used to detect the expression level of at least one IRG and/or variant thereof. One of skill in the art will appreciate that the macroarray can include a number of test probes that specifically hybridize to the expressed nucleic acid which is to be detected and, optionally, one or more control probes. Test probes can include oligonucleotides that range in size (e.g., between about 5 and 50 nucleotides) and have sequences complimentary to particular subsequences of the genes whose expression they are designed to detect. Thus, the test probes may be capable of specifically hybridizing to a target nucleic acid. Examples of control probes that may be included as part of the macroarray can include normalization controls, expression level controls, and mismatch controls.

In another example of the present invention, a macroarray as described in Example 2 (below) can be used to detect the expression level of at least about 4 of the genes listed in Table 1. Detecting the expression level of at least about 4 genes (e.g., 4 genes) may be advantageous for several reasons. To conduct a quantitative test (e.g., qPCR), for example, selection of a limited number of genes in a multiplex array may be useful for practical reasons (e.g., volume and number of reagents needed, etc.). Additionally, selection of at least about 4 genes can be done to optimize the discriminating ability (i.e., area under an ROC curve) using the random forest model of the present invention.

The IRGs comprising the macroarray may be represented by about 166 human cDNAs. Briefly, the protocol for spotting DNA on the macroarray membrane, probe labeling, and hybridization can begin by isolating about 5 μg of total RNA ex vivo from whole blood. cDNA probes can then be generated by reverse transcription using SUPERSCRIPT II in the presence of ³²PdCTP (INVITROGEN, Carlsbad, Calif.). Residual RNA can be hydrolyzed by alkaline treatment at about 70° C. for about 20 minutes, after which cDNA can be purified using G50 columns (GE Healthcare, Buckingham-shire, UK). Probes can then be hybridized overnight to the macroarray membrane in about 10 milliliters of hybridization buffer, followed by wash with low and high stringency buffers. Next, the macroarray can be exposed to intensifying phosphor screens for about two days, followed by scanning with STORMIMAGER (MOLECULAR DYNAMICS, Sunnyvale, Calif.).

Prior art methods frequently employ high density oligonucleotide microarrays to characterize genes regulated by IFN-β. Such methods may be useful for identifying novel IFN-β regulated genes, but results are not readily quantified, and the technique is therefore less suitable for analyzing longitudinal, differential IRG regulation. Unlike the high density microarrays of the prior art, the macroarray of the present invention can include about 166 IRGs selected from previous microarray experiments (see, e.g., Schlaak, J. F. et al., Biol. Chem. 277:49428-49437, 2002; and Rani, M. R. S. et al., Ann. N.Y. Acad Sci. 1182:58-68, 2009) that validated the macroarray for other disease indications (e.g., IFN-α treatment for hepatitis C virus) and confirmed that: the microarray is reproducible, sensitive, and quantitative. Advantageously, the relatively small number of genes detectable by the macroarray of the present invention provides a focused and quantitative assay for assessing IFN-β-regulated gene expression.

At Step 18, the measured gene expression level can be analyzed to determine the efficacy of IFN-β therapy. For example, the measured level of gene expression can be compared to the gene expression level of a control (e.g., one or more subjects without MS). In one example of the present invention, an increased or decreased expression level of at least about 4 of the genes listed in Table 1 and/or variants thereof as compared to the control may indicate that the subject will respond poorly to IFN-β therapy. In addition to exhibiting an increased or decreased level of gene expression, poor responders can also demonstrate continual neurological deterioration despite therapy. Methods for assessing neurological deterioration in subjects with MS are known in the art and can include, for example, quantitative MRI analysis, the Expanded Disability Status Scale (EDSS) (e.g., an EDSS score increased by at least about 0.5 may be indicative of neurological deterioration), and the Multiple Sclerosis Functional Composite.

In another example of the present invention, an increased or decreased expression level of at least one (e.g., about 4) of the following genes and/or variants thereof (as compared to control) may indicate that the subject will respond poorly to IFN-β therapy: 2-5OAS; Adaptin; Akt-2; APOL3; ATF 2; Bad; Bcl-2; BST2; C1-INH; Clorf29; C1r; C1S; Caspase 1; Caspase 7; Caspase 9; CCR1; CD3e; CEACAM; c-myc; COMT; CREB; CXCL11; CXCR4; CYB56; DDX17; Def-a3; Elastase 2; Fas-L; FK506; FLJ20035; G1P3; Gadd45; GATA 3; GBP2; HLADP; HLADRA; Hou; HPAST; Hsf1; Hsp90; IDO; IFI16; 1FI-17; IFN-44; 1F160; IFIT1; IFIT2; IFIT5; IFITM2; IFIM3; IFN-17; IFNAR1; IFNAR2; IFNGR1; IFNGR2; IL15; IL18 BP; IL1RN; IL2; IL2Rg; IL6; Int-6; IP-10; IRF2; ISG15-L; ISG20; 1SGF3g; L1CAM; MAP2K3; MAP2K4; MAP3K14; MAP3K3; MAP3K4; MAP3K7; MAP4K1; MAPK13; MAPK7; Met-onto; MMP-1; MMP-9; MT1H; MT1X; MT2A; MX1; NF-IL-6; NFκB; NMI; NT5e; OASL; P4HA1; p53; p57Kip2; PA1-1; PDK1; PDK2; PI3K; PKR; plectin; PLSCR1; PSMB9; RCNI; RGS2; RHO GDP; RIG-1; SERPIN; SNN; SOCS-1; STAT1; STAT2; STAT4; TFEC; TGFbR2; TGFbR3; TIMP-1; TNF-α; TNFAIP6; TOR1B; TRAIL; UBE2L6; USP18; VegFC; Viperin; and WARS.

In another example of the present invention, an increased or decreased expression level of at least one (e.g., about 4) of the following genes and/or variants thereof (as compared to control) may indicate that the subject will respond poorly to IFN-β therapy: TRAIL; RIG-1; 2-5OAS; STAT1; PI3-kinase; IL-15; IP-10; MMP-1; P4HA1; caspase 7; PDK2; ATF-2; TNF-α; RGS2; SNN; hsp90; c-myc; A1-AT; HLA-DRA; COMT; NFκB; HLA-DP; TIMP-1; CXCR4; and IL-2.

In another example of the present invention, an increased expression level of at least one (e.g., about 4) of the following genes and/or variants thereof (as compared to a control) may indicate that the subject will respond poorly to IFN-β therapy: TRAIL; RIG-1; 2-5OAS; STAT1; PI3-kinase; IL-15; IP-10; MMP-1; P4HA1; caspase 7; PDK2; ATF-2; TNF-α; and RGS2.

In another example of the present invention, a decreased expression level of at least one (e.g., about 4) of the following genes and/or variants thereof (as compared to a control) may indicate that the subject will respond poorly to IFN-β therapy: SNN; hsp90; c-myc; A1-AT; HLA-DRA; COMT; NFκB; HLA-DP; TIMP-1; CXCR4; and IL-2.

Another aspect of the present invention can include determining whether a subject with MS should be treated with a therapeutic agent other than IFN-β. Where, for example, a subject with MS has an increased or decreased expression level of at least one IRG and/or variant thereof (e.g., at least about 4 of the genes listed in Table 1) as compared to a control, the subject can be treated with a therapeutic agent other than IFN-β. MS therapies other than IFN-β are known in the art and can include, for example, glatiramer acetate, mitoxantrone, and natalizumab, as well as alternative therapies (e.g., vitamin D). Other MS therapies can include those currently under clinical investigation for the treatment of MS, such as of aIemtuzumab, daclizumab, inosine, BG00012, fingolimod, laquinimod, and NEUROVAX. Methods for treating subject with MS according to the present invention are described in greater detail below.

At Step 20, the method 10 can optionally include administering a dose of IFN-β to a subject with MS prior to obtaining the biological sample. The IFN-β dose can be delivered as a single preparation, which may reduce noise in the gene expression measure (i.e., at Step 16). Examples of IFN-β doses that can be administered to a subject with MS include IFN-β-1a (e.g., AVONEX, REB1F) and IFN-β-1b (e.g., BETASERON, EXTAVIA). The IFN-β dose can be administered via any known route, such as intravascular injection.

Following administration of the IFN-β dose to the subject, at least one biological sample can be obtained (as described above). The biological sample can be obtained at one or more time points. For example, a whole blood sample can be obtained from a subject with MS about 12 hours after administration of an IFN-β dose. It should be appreciated that additional doses of IFN-β can be administered to a subject following a first IFN-β dose. For example, a first dose of IFN-β can be administered to a subject, followed by collection of a biological sample about 12 hours after the first dose and then a second dose of IFN-β at about 6 months, again followed by collection of a biological sample. After obtaining the biological sample, at least one nucleic acid can be isolated from the sample (as described above). As also described above, the level of expression of at least one IRG and/or variant thereof can then be determined using, for example, a macroarray.

Once the expression level of the at least one IRG and/or variant thereof has been determined, the expression level can be analyzed (as described above). For example, the measured level of gene expression can be compared to the gene expression level of a control. The control can be isolated from one or more subjects without MS, obtained from a subject who has not been treated with IFN-β, or taken from a subject before being treated with IFN-β. Where the level of measured gene expression is increased or decreased in at least about 4 of the genes listed in Table 1 (as compared to the control), for example, the subject may respond poorly to IFN-β therapy.

Although IFN-β is the most commonly used disease-modifying treatment for MS, its mechanisms of action are not well understood and there are no biological markers that can guide individualized therapy. Based on the discovery that an exaggerated molecular response to IFN-β injections in subjects with MS is a marker for a subset of subjects in whom innate immune responses drive pathogenesis, the present invention advantageously provides a method 10 for identifying the minority of subjects destined for poor responder status on IFN-β therapy. As discussed in greater detail below, the present invention thereby enables the tailoring of disease-modifying therapy for individual subjects with MS.

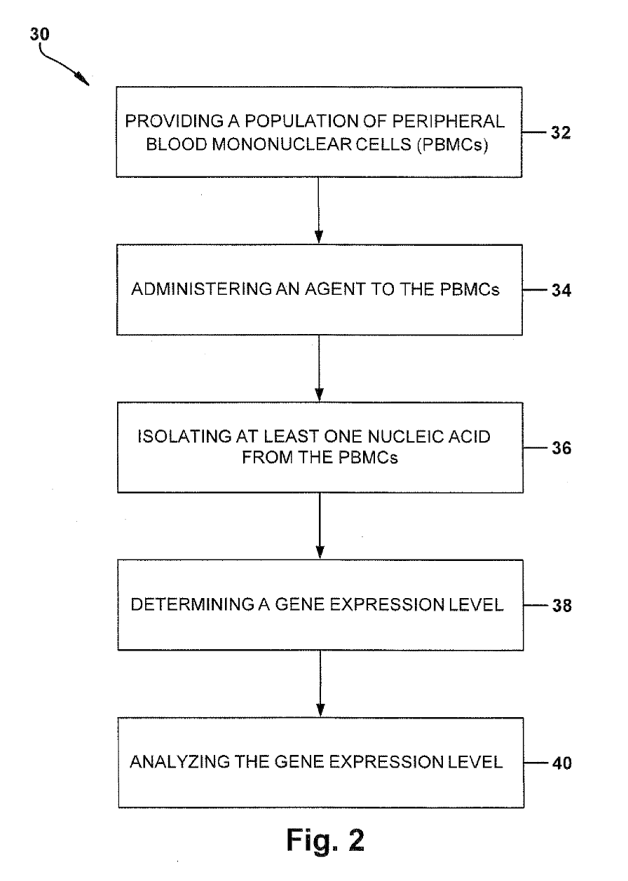

FIG. 2 illustrates another aspect of the present invention comprising a method 30 for screening an agent that can be used to treat MS. The method 30 can comprise the steps of: providing a population of peripheral blood mononuclear cells (PBMCs) from a subject with MS (Step 32); administering an agent to the PBMCs (Step 34); isolating at least one nucleic acid from the PBMCs (Step 36); determining the gene expression level of at least one IRG and/or variant thereof (Step 38); and analyzing the measured gene expression level (Step 40).

At Step 32, a population of PBMCs can be obtained from a subject that has MS and is a poor responder to IFN-β therapy. A determination of whether the subject is a poor responder can be made according to the method 10 described above. For example, a subject with MS that has an increased or decreased expression level of at least one IRG and/or variant thereof (e.g., about 4 of the genes listed in Table 1) as compared to a control may be characterized as a poor responder. One skilled in the art will appreciate that there are several methods for isolating PBMCs. For example, PBMCs can be isolated from a whole blood sample using different density gradient centrifugation procedures. Typically, anti-coagulated whole blood can be layered over a separating medium and then centrifuged. At the end of the centrifugation step, several layers can be visually observed (from top to bottom): plasma/platelets; PBMCs; separating medium; and erythrocytes/granulocytes. The PBMC layer can be removed and washed to get rid of any contaminants (e.g., red blood cells). After washing, cell type and cell viability can be confirmed using methods known in the art. The PBMCs can then be cultured ex vivo for a time and under conditions sufficient to promote a substantially confluent cell layer.

At Step 34, at least one agent can be administered to the population of PBMCs. Agents that may be administered to the population of PBMCs can include any biological moiety, compound, or drug that may be a candidate for MS therapy. Examples of such agents can include biologics, pharmaceutical compounds, polypeptides, proteins, nucleic acids, and small molecules.

At Step 36, at least one nucleic acid can be isolated from the population of PBMCs. Methods for isolating nucleic acids from cell populations are known in the art. For example, RNA can be isolated from the population of PBMCs using a known RNA extraction assay.

As described above, the level of expression of at least one IRG and/or variant thereof (e.g., about 4 of the genes listed in Table 1) can be determined at Step 38. For example, a macroarray can be used to detect gene expression levels.

Once the expression level of the at least one IRG and/or variant thereof (e.g., about 4 of the genes listed in Table 1) has been determined, the measured expression level can be analyzed at Step 40 (as described above). For example, the measured level of gene expression can be compared to the gene expression level of a control. Where the measured level of gene expression is increased or decreased (as compared to a control), the administered agent may not be a candidate for MS therapy. Conversely, where the level of gene expression is not increased or decreased (as compared to the control sample), the administered agent may be a candidate for MS therapy.

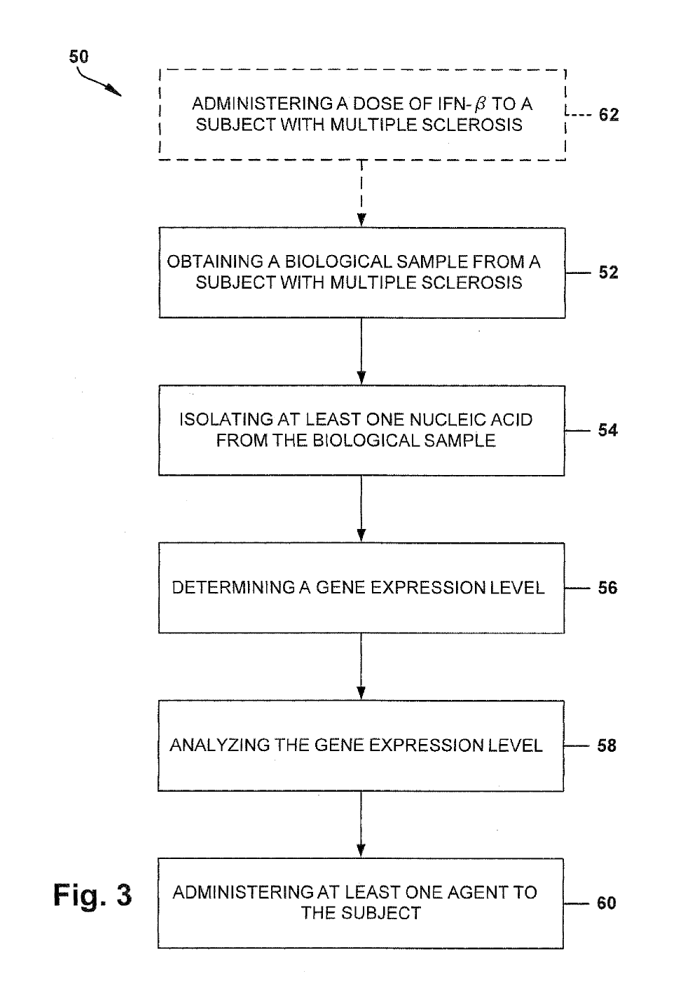

FIG. 3 illustrates another aspect of the present invention comprising a method 50 for treating a subject with MS. The method 50 can include the steps of: obtaining a biological sample from a subject with MS (Step 52); isolating at least one nucleic acid from the biological sample (Step 54); determining the gene expression level of at least one IRG and/or variant thereof (Step 56); analyzing the measured gene expression level (Step 58); and administering at least one agent to the subject (Step 60). Optionally, the method 50 can include administering a dose of IFN-β to a subject with MS prior to obtaining the biological sample (Step 62).

At Step 52, at least one biological sample can be obtained from a subject with MS. As described above, for example, the biological sample can include a whole blood sample obtained using a syringe needle from a vein of the subject.

At Step 54, at least one nucleic acid can be isolated from the biological sample (as described above). For example, RNA can be isolated from a whole blood sample using the PAXGENE RNA blood extraction kit.

Next, the level of expression of at least one IRG and/or variant thereof can be determined at Step 56. As described above, for example, a hybridized macroarray can be used to detect gene expression levels in at least about 4 of the genes listed in Table 1.

Once the expression level of the at least one IRG and/or variant thereof has been determined, the measured gene expression level can be analyzed at Step 58 (as described above). For example, the measured level of gene expression can be compared to the gene expression level of a control. Where the measured level of gene expression is increased or decreased (as compared to a control), the subject may be a poor responder to IFN-β therapy. Conversely, where the level of gene expression is not increased or decreased (as compared to the control sample), the subject may be a candidate for IFN-β therapy.

At Step 60, a therapeutically effective amount of at least one agent can be administered to the subject. The particular agent administered to the subject will depend upon the subject's previously-determined responder status. For example, if the subject is a poor responder, then a therapeutically effective amount of an agent other than IFN-β, such as natalizumab can be administered to the subject. Conversely, if the subject is a poor responder, then a therapeutically effective amount of an agent, such as IFN-β can be administered to the subject. It will be appreciated that the type of treatment, dosage, schedule, and duration of treatment can vary, depending upon the severity of pathology and/or the prognosis of the subject. Those of skill in the art are capable of adjusting the type of treatment with the dosage, schedule, and duration of treatment. Advantageously, the method 50 provides a regimen for treating subjects with MS without exposing them to unnecessary medicaments, which, in turn, may be highly beneficial in terms of saving unnecessary costs to the health care system.

It will also be appreciated that the method 50 can optionally include the step of administering a dose of IFN-β to a subject with MS prior to obtaining the biological sample (as discussed above) at Step 62.

It will be further appreciated that the present invention can alternatively include protein or polypeptide isolation and detection techniques as part of the method 10, 30, and 50. For example, known techniques can be used to isolate and detect proteins, polypeptides, and/or variants thereof encoded by the IRGs and/or variants thereof of present invention. To do so, a biological sample can be obtained from a subject with MS (as described above). Next, the biological sample can be subjected to a known technique for isolating a protein, polypeptide, and/or variant thereof encoded by an IRG and/or variant thereof of present invention. See, e.g., Protein Purification Protocols, Humana Press (1996). The isolated protein, polypeptide, and/or variant thereof can then be detected using one or a combination of known techniques, such as protein microarray, immunostaining, immunoprecipitation, electrophoresis (e.g., 2D or 3D), Western blot, spectrophotometry, and BCA assay. Following detection of the protein, polypeptide, and/or variant thereof, the level of the protein, polypeptide, and/or variant thereof can be analyzed. Where the level of the protein, polypeptide, and/or variant thereof is increased or decreased (as compared to a control sample), the subject may be a poor responder to IFN-β therapy. Conversely, where the level of the protein, polypeptide, and/or variant thereof is not increased or decreased (as compared to the control sample), the subject may be a candidate for IFN-β therapy.

The following examples are for the purpose of illustration only and are not intended to limit the scope of the claims, which are appended hereto.

### Example 1

### Methods

### Clinical Protocol

The Cleveland Clinic (CC) Institutional Review Board approved the study. All subjects provided written informed consent. Subjects were eligible if they had clinically isolated syndrome (CIS) or relapsing-remitting MS, were initiating intramuscular IFN-β-1a treatment, were previously treatment-naïve, and were followed at CC MS Center. Ninety-nine subjects were enrolled. Each patient was examined at baseline, 6, 12, and 24 months. At 3 and 18 months, patients were contacted by phone to assess treatment compliance and ascertain side effects. At the baseline visit, 6, and 24 months, blood was collected in a clinical research unit for IRG analysis immediately before and exactly 12 hours after an IFN-β injection, and the patients had standardized brain MRI scans for quantitative assessment of lesions and brain atrophy. At each visit, patients had neurological exams to determine the Kurtzke Expanded Disability Scale Score (Kurtzke, J. F., Neurology 33:1444-1452, 1983), the Multiple Sclerosis Functional Composite score (Rudick, R. A. et al., Mult. Scler. 8:359-365, 2002), and history of intercurrent relapses or illness; they were also given a structured questionnaire to characterize flu-like symptoms, muscle aches, chills, fatigue, headache, and loss of strength. Serum was tested for IFN-neutralizing antibodies at 6 and 24 months.

### MRI Analysis

The MRI acquisition included a T2-weighted fluid-attenuated inversion recovery (FLAIR) image, T2- and proton density-weighted dual echo fast spin echo images, and T1-weighted spin echo images acquired before and after injection of standard dose gadolinium (0.1 mmol/kg). Images were analyzed using software developed in house to determine brain parenchymal fraction (BPF), T2 lesion volume, T1 hypointense lesion volume, gadolinium-enhancing lesion volume and number, the number of new T2 lesions, and the number of enlarging T2 lesions. BPF was calculated from FLAIR images using fully-automated segmentation software (Rudick, R. A. et al., J. Neuroimmunol. 93:8-14, 1999). Details of the lesion analysis methods have been previously described (Cohen, J. A. et al., Mult, Scler. 14:370-382, 2008). Briefly, T2 hyperintense lesions were automatically segmented in the FLAIR and T2/PD images and visually verified using interactive software to correct misclassified lesions. Six-month follow-up images were registered to baseline, and intensity normalized. Baseline T2 lesion masks were applied to the co-registered 6-month images to identify persistent lesions. The baseline images were then subtracted from the registered, intensity normalized 6-month images to automatically identify new and enlarging T2 lesions at 6 months. New and enlarging T2 lesions were visually verified using interactive software to generate the final counts.

### RNA Isolation

RNA was extracted ex-vivo from blood using PAXGENE RNA blood extraction kit (PreAnalytix, Switzerland) as per the manufacturer's instructions and concentrated by ethanol precipitation. RNA quality and quantity was assessed by spectrophotometry (absorbance ratios of 280/260 nm) and additional visualization by agarose gel electrophoresis. RNA samples were stored at −80° C.

### Genes Analyzed Using Macroarray

The detailed methodology for cDNA macroarray analysis was performed as described (Schlaak, J. F. et al., J. Biol. Chem. 277, 49428-49437, 2002; Rani, M. R. S. et al., Ann. N.Y. Acad Sci. 1182:58-68, 2009). IRGs on the custom macroarray were represented by 166 human cDNAs selected from the Unigene database. A list of the names of all genes on the macroarray with GenBank accession numbers is shown in Table 1.

TABLE 1
Name and GenBank accession numbers for the 166 type 1
interferon responsive genes selected for the customized
macroarray

| Gene | Accession No. |
|---|---|
| 2-5OAS | NM_002534 |
| a1-AT | K01396 |
| ADAM17 | U69611 |
| Adaptin | AF068706 |
| Akt-1 | NM_005163 |
| Akt-2 | M77198 |
| APOL3 | AA971543 |
| ATF 2 | X15875 |
| Bad | U66879 |
| Bax | U19559 |
| Bcl-2 | M14745 |
| BST2 | D28137 |
| C1-1NH | NM_000062 |
| C1orf29 | NM_005951 |
| C1r | NM_001733 |
| C1S | J04080 |
| Caspase 1 | M87507 |
| Caspase 7 | U67319 |
| Caspase 9 | U60521 |
| CBFA | NM_004349 |
| CCR1 | L09230 |
| CCR5 | U54994 |
| CD14 | NM_000591 |
| CD3e | NM_012099 |
| CEACAM | NM_001712 |
| c-fos | NM_005252 |
| c-myc | L00058 |
| Collagen | J03464 |
| COMT | M58525 |
| CREB | NM_004379 |
| CXCL1I | NM_005409 |
| CXCR4 | AF005058 |
| CYB56 | NM_007022 |
| Cyp19 | M28420 |
| DDX17 | U59321 |
| Def-a3 | NM_005217 |
| Destrin | S65738 |
| Elastase 2 | M34379 |
| F-actin | U56637 |
| Fas-L | U08137 |
| FK506 | AF038847 |
| FLJ20035 | AK000042 |
| G1P3 | NM_002038 |
| Gadd45 | M60974 |
| GATA 3 | X58072 |
| GBP2 | M55543 |
| Gran B | M17016 |
| HLADP | M83664 |
| HLADRA | J00194 |
| HLAE | X56841 |
| Hou | U32849 |
| HPAST | AF00144 |
| Hsf1 | M64673 |
| Hsp90 | X15183 |
| IDO | NM_002164 |
| IF1I6 | M63838 |
| IFI-17 | J04164 |
| IFI35 | U72882 |
| IFI44 | D28915 |
| IFN-44 | D28915 |
| IFI60 | AF083470 |
| IFIT1 | M24594 |
| IFIT2 | NM_001547 |
| IFIT4 | NM_001549 |
| IF1T5 | NM_012420 |
| IFITM2 | NM_006435 |
| IFITM3 | X57352 |
| IFN-17 | M13755 |
| IFN-9/27 | J04164 |
| IFNAR1 | J03171 |
| IFNAR2 | L42243 |
| IFNGR1 | J03143 |
| IFNGR2 | U05875 |
| IkBa | M69043 |
| IL15 | U14407 |
| IL18 BP | AB019504 |
| ILIRN | NM_000577 |
| IL2 | NM_000586 |
| IL2Rg | NM_000206 |
| IL6 | X04602 |
| IL8Rb | NM_001557 |
| iNOS | U20141 |
| Int-6 | U62962 |
| integ-b-6 | NM_000888 |
| IP-10 | X02530 |
| IRF4 | U52682 |
| IRF1 | L05072 |
| IRF2 | X15949 |
| IRF7 | U73036 |
| ISG15-L | M13755 |
| ISG20 | NM_002201 |
| ISGF3g | M87503 |
| JUN | J04111 |
| L1CAM | M74387 |
| L-Selectin | M25280 |
| MAP2K3 | NM_002756 |
| MAP2K4 | L36870 |
| MAP3KI1 | NM_002419 |
| MAP3KI4 | NM_003954 |
| MAP3K3 | U78876 |
| MAP3K4 | NM_005922 |
| MAP3K7 | NM_003188 |
| MAP4KI | NM_007181 |
| MAPK13 | AF004709 |
| MAPK7 | NM_002749 |
| Met-onco | NM_000245 |
| MIP-Ib | NM_002984 |
| MMP-1 | M13509 |
| MMP-9 | NM_004994 |
| MTIH | NM_005951 |
| MT1X | NM_005952 |
| MT2A | NM_005953 |
| MX1 | M33882 |
| MX2 | M30818 |
| NF-IL-6 | X52560 |
| NFkB | M58603 |
| NMI | Y00664 |
| NT5e | X55740 |
| OASL | NM_003733 |
| P4HA1 | M24486 |
| p53 | M14694 |
| p57Kip2 | U22398 |
| p70 K | M60724 |
| PAI-1 | M16006 |
| PDGF-a | X06374 |
| PDK1 | Y15056 |
| PDK2 | NM_002611 |
| PGK | V00572 |
| PI3K | NM_006219 |
| PIAS | AF077954 |
| PIAS1 | AF077951 |
| Pig7 | AF010312 |
| PKR | NM_002759 |
| plectin | U53204 |
| PLSCR1 | AF098642 |
| PSMB9 | X66401 |
| Raf | X03484 |
| RCNI | D42073 |
| RGS2 | NM_002923 |
| RHO GDP | L20688 |
| Ribonuc | NM_003141 |
| R1G-1 | AF038963 |
| SERPIN | NM_000295 |
| Smad1 | U59423 |
| SNN | NM_003498 |
| SOCS-1 | N91935 |
| SOCS2 | AF020590 |
| SSA1 | NM_003141 |
| STAT1 | M97935 |
| STAT2 | M97934 |
| STAT4 | L78440 |
| STAT5A | L41142 |
| TAP1 | X57522 |
| TFEC | NM_012252 |
| TGFbR2 | D50683 |
| TGFbR3 | L07594 |
| TIMP-1 | M59906 |
| TNT-a | X01394 |
| TNFAIP6 | NM_007115 |
| TOR1B | NM_014506 |
| TRAIL | U37518 |
| UBE2L6 | NM_004223 |
| USP18 | NM_017414 |
| VegFC | U43142 |
| Viperin | AF026941 |
| WARS | X62570 |

These Type 1 IFN IRGs were identified by microarray analysis of fibrosarcoma, epithelial or endothelial cell lines treated either with IFN-α or IFN-β (Schlaak, J. F. et al., J. Biol. Chem. 277, 49428-49437, 2002; Rani, M. R. S. et al., Ann. N. Y Acad Sci. 1182: 58-68, 2009). All the genes were known IRGs.

The protocol for spotting DNA on the membrane, probe labeling and hybridization has been described previously, with modifications as follows (Schlaak, et al., J. Biol. Chem. 277, 49428-49437, 2002; Rani, M. R. S. et al., Ann. N.Y. Acad Sci. 1182:58-68, 2009). Total RNA, 5 μg, isolated ex vivo from blood was used for generating radiolabeled cDNA probes by reverse transcription with SUPERSCRIPT II (Invitrogen, Carlsbad, Calif.) in the presence of ³²PdCTP. Residual RNA was hydrolyzed by alkaline treatment at 70° C. for 20 minutes after which cDNA was purified using G50 columns (GE Healthcare, Buckingham-shire, UK). Preparation of macroarrays and hybridization of radioactive cDNA were conducted as described previously (Schlaak, J. F. et al., J. Biol. Chem. 277, 49428-49437, 2002; Rani, M. R. S. et al., Ann. N.Y. Acad. Sci. 1182:58-68, 2009). Radioactivity bound to the membrane was quantitated, and used to calculate IR of the ISGs.

To minimize variability, each patient's samples at baseline (0 months) and 6 months were processed in a single batch experiment (total of 4 membranes).

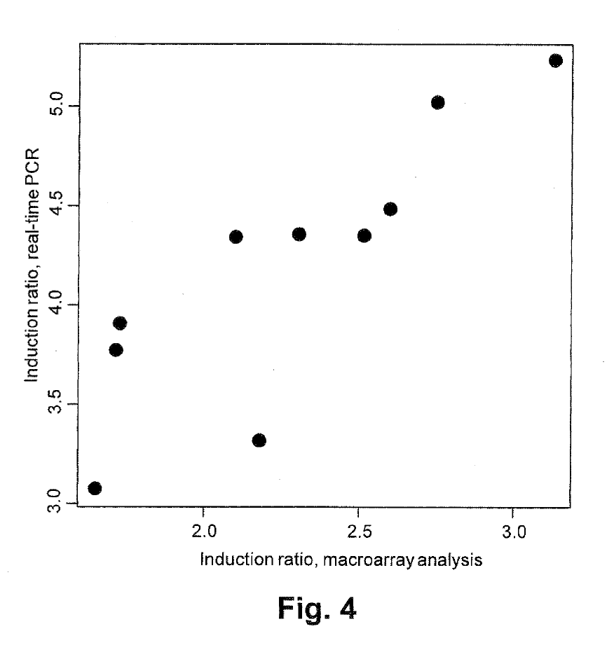

Induction ratios (IRs) generated using the custom cDNA macroarray were validated using real-time quantitative PCF for 5 genes: OASL (accession number NM003733); TRAIL (U37518); IFI44 (D28915); HLADRA (J00194); and TIMP-1 (M59906). Spearman correlation coefficients for the correlations between the rt-PCR and macroarray data for OASL, TRAIL, IFI44, HLADRA, and TIMP-1 were 0.92, 0.75, 0.36, 0.72, and 0.54 respectively. FIG. 4 shows the IRs and correlations obtained for OASL.

### Statistical Analysis

Poor response to IFN-β was based on quantitative MRI analysis, comparing the MRI at the 6 month visit with baseline. Poor response was defined as the occurrence of ≧3 new lesions. Differences in baseline characteristics between good and PR groups were compared using t-tests or Fisher's exact tests, as appropriate. A Poisson regression was used to test group differences in the number of induced IRGs with IRs ≧2.0 at the baseline injection. Pearson correlation coefficients of log 2 transformed IRs at first injection compared with 6 months were computed for 85 patients. Baseline, 6 months, and 24 months pair-wise correlations were computed for 10 randomly selected patients.

Demographic and baseline MRI adjusted least-square means (LS means) of the log 2-transformed IRs were computed and compared between response groups by ANCOVA. The covariates were age, sex, presence of gadolinium-enhancing lesions, and T2 volume. To investigate whether the groups differed with respect to the overall distribution of the magnitude of response to IFN-β, density plots of the 166 IRGs LS means were generated for the groups, comparing IRs at baseline and 6 months with responder status. The proportion of genes showing greater response (LS mean: PRs >GRs in up-regulated genes, or PRs <GRs in down-regulated genes) in PRs was tested (one-sided) with a binomial proportion test assuming a null hypothesis of proportion ≦0.5.

To further investigate whether IRGs could discriminate PRs from GRs, the IRGs at baseline that best discriminated between poor and GRs were identified as follows. First, the univariately differential IRGs were selected, then a random forest technique was used to select genes and build the prediction model. The best 25 IRGs were selected based on the rank of a Monte-Carlo based sum-of-rank estimate of the variable importance obtained from 1000 random forest simulations. The estimated ROC curves based on these 25 genes in classifying patients to their correct response group were compared with and without baseline T2 volume in the prediction models.

### Results

### Research Subjects

Ninety-nine subjects were entered into the longitudinal study. Eighty-five remained in the protocol and continued to take intramuscular IFN-β-1a for at least 6 months. Of the 14 patients who did not complete the planned 6-month macroarray analysis, 12 discontinued IFN-β-1a, whereas sample hybridization was unsuccessful in the other 2, either at first injection or 6 months. Baseline demographic and disease characteristics did not significantly differ between the 85 patients who completed the first 6 study months, and the 14 who did not (data not shown). For all other analyses, only the 85 patients who completed the first 6 months were included. Among these 85, 32% had clinically isolated syndromes with multiple brain MRI lesions, and 68% had relapsing-remitting MS. The mean age was 35.7 years; mean MS disease duration was 2.4 years; 65% were women; and 91% were white. At 6 months, 15 (18%) of the study subjects were classified as PRs based on the pre-determined MRI definition. Table 2 lists baseline characteristics for PRs, GRs and the entire population.

TABLE 2
Comparison of baseline characteristics between patients
with good vs poor response to IFN-β treatment*

| | Good responders | Poor responders | All patients | P-value |
|---|---|---|---|---|
| Characteristic | (n = 70) | (n = 15) | (n = 85) | (GR vs PR) |
| Age (years) | 36.3 (9.4) | 33.0 (11.2) | 35.7 (9.8) | 0.30 |
| Symptom duration (years) | 2.5 (3.0) | 1.2 (1.7) | 2.4 (2.9) | 0.39 |
| % Female | 69% | 47% | 65% | 0.11 |
| % White | 93% | 80% | 91% | 0.14 |
| % CIS/% RRMS | 34%/66% | 20%/80% | 32%/68% | 0.37 |
| EDSS | 1.6 (1.0) | 1.6 (1.2) | 1.6 (1.0) | 0.91 |
| MSFC score | 0.39 (0.48) | 0.19 (0.41) | 0.35 (0.47) | 0.10 |
| % with Gad-enhancing lesions | 24.3% | 53.3% | 29.4% | 0.03 |
| Gad-enhancing lesion volume | 0.097 (0.38) | 0.44 (0.72) | 0.16 (0.47) | 0.09 |
| T2 volume | 3.0 (3.7) | 5.8 (3.9) | 3.5 (3.8) | 0.02 |
| T1 BH Volume | 0.55 (0.75) | 0.87 (0.82) | 0.61 (0.77) | 0.19 |
| BPF | 0.858 (0.014) | 0.859 (0.013) | 0.859 (0.014) | 0.79 |

*All values are mean ± SD, unless otherwise indicated.
CIS = clinically isolated syndrome;
RRMS = relapsing-remitting multiple sclerosis;
EDSS = Expanded Disability Scale Score;
MSFC = Multiple Sclerosis Functional Composite;
Gad = gadolinium;
BH = black hole;
BPF = brain parenchymal fraction.

The two groups were similar at baseline on all characteristics except that a higher proportion of PRs had gadolinium-enhancing lesions at baseline, and they had greater T2 lesion volumes.

### IRG Response to First Injection and Stability Over Time

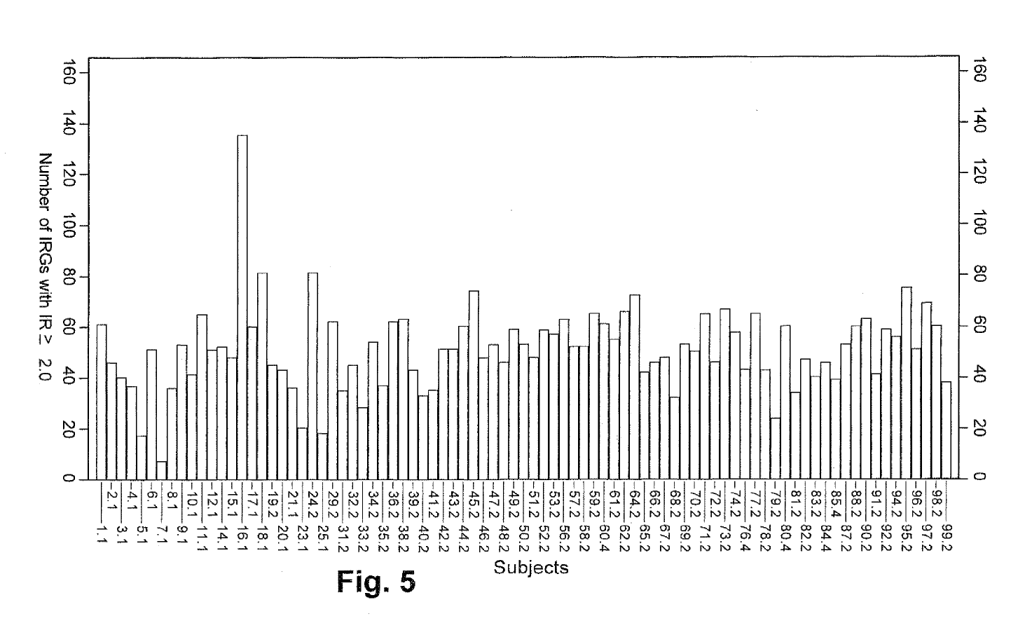

An IR>2.0 defined induction of an IRG, as assays in healthy subjects not receiving IFN-β injections failed to show IRGs that varied more than 1.5-fold in assays separated by 12 or 24 hours. The number of induced IRGs at the first IFN-β injection varied among patients, ranging from 7 to 135, with no relationship between IFN-β responder status and number of induced genes (P=0.76) (FIG. 5). Similarly, the pattern of response to the initial IFN-β injection varied considerably between patients (Rani, M. R. S. et al., Ann. N.Y. Acad Sci. 1182:58-68, 2009).

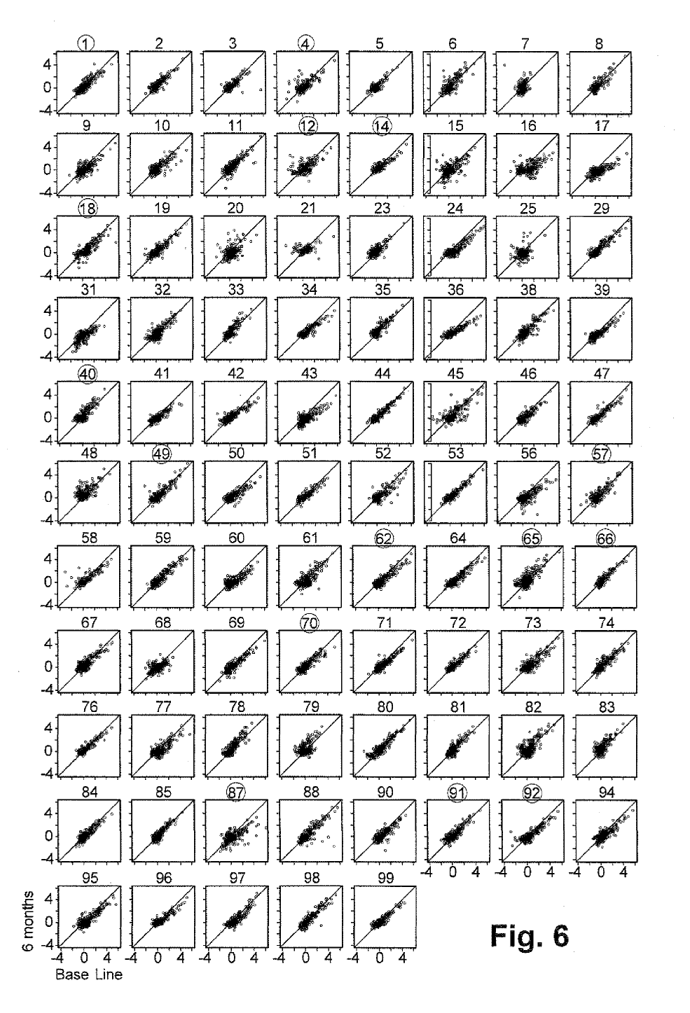

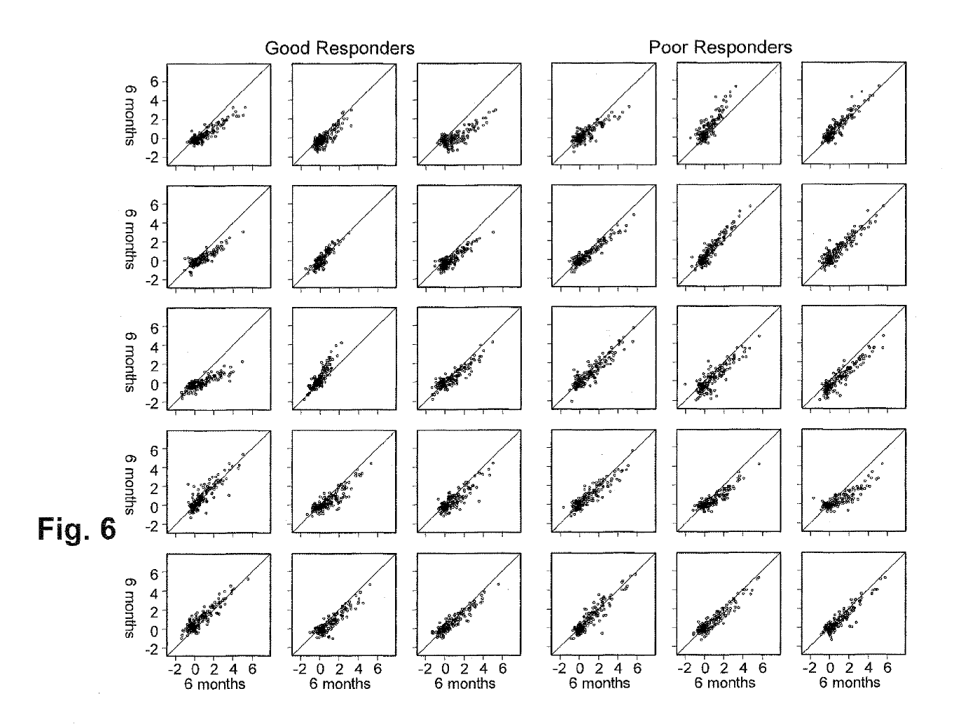

Despite considerable inter-individual variability in the pattern and magnitude of IRG response after the first IFN-β-1a injection, the response was stable over time for individual subjects. FIG. 6 shows the IRs at first injection (x-axis) plotted against IRs at 6 months (y-axis) for all 85 patients. The molecular response to IFN-β injections was remarkably stable for almost all patients. There were three exceptions—subject 7 (top row, 7th from left) and subject 25 (third row, first from the left) had viral infections at the baseline dose and so had little or no IRG induction at first injection, due to high pre-injection IRG expression levels. Both subjects responded to IFN-β injection at 6 months. Subject 21 (second row, 9th from left) developed high titer neutralizing antibodies to IFN-β detected at 6 months. Subject 21 responded briskly to the first IFN-β injection, but minimally at 6 months. Neutralizing antibody testing of all other subjects was negative at 6 months.

Excluding those three subjects, IRs at first injection strongly correlated with IRs at 6 months for individual patients [Pearson correlation coefficient mean (±SD)=0.81±0.11]. The mean correlation coefficient for the 15 PR subjects (study numbers 1, 4, 12, 14, 18, 40, 49, 57, 62, 65, 66, 70, 87, 91, and 92) was 0.81±0.10, compared with a mean of 0.81±0.11 for the 67 GR patients (excluding subjects 7, 21, 25).

The IRG analysis was repeated at 24 months for 10 randomly selected patients (5 PRs and 5 GRs) (FIG. 7). For these 10 subjects, IRs strongly correlated between baseline and 6 months (r=0.86); between 6 months and 24 months (r=0.82); and between baseline and 24 months (r=0.85). Correlation coefficients were similar for the 5 PRs and 5 GRs.

These results suggested that PR status could not be attributed to either the magnitude of the molecular response to IFN-β (FIG. 5) or attenuation of the molecular response to IFN-β over time (FIGS. 6-7).

### IRG Response in Good Vs Poor IFN-β Responders

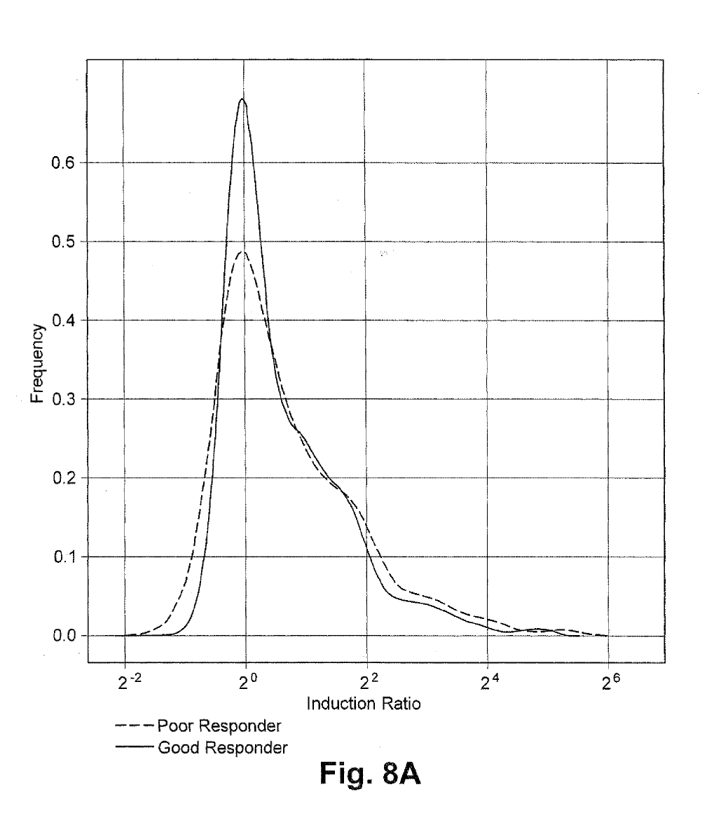

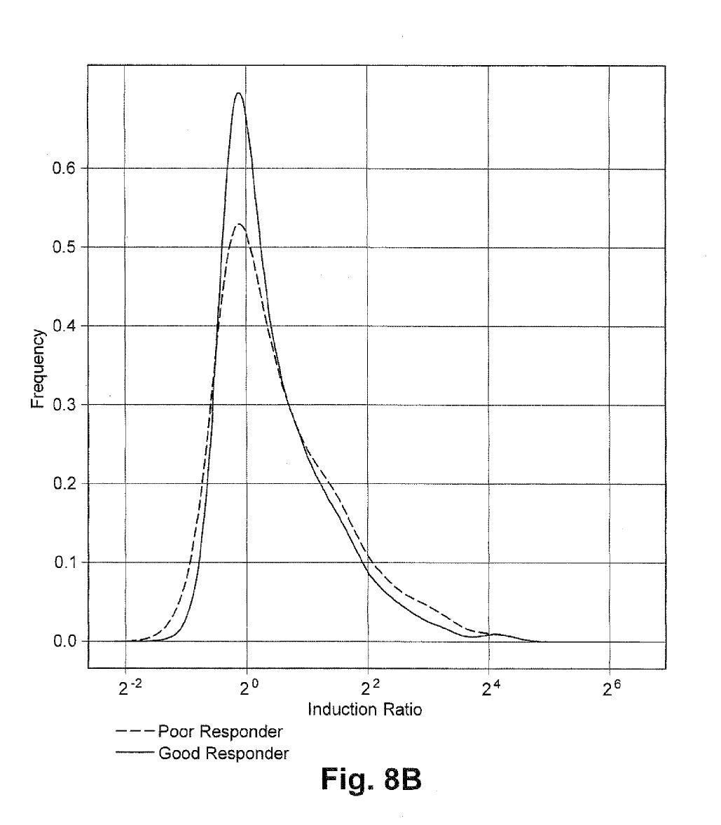

The biological effects of IFN-β are accounted for by the activities of the IRG protein products (Borden, E. C. et al., Nat. Rev. Drug Discov. 6:975-990, 2007). We addressed whether the characteristics of the molecular response to IFN-β might explain PR status, either by revealing induction of deleterious inflammatory gene products (Wandinger, K. P. et al., Ann. Neural. 50:349-357, 2001) or selective failure of expression of beneficial genes (Wandinger, K. P. et al., Lancet 361:2036-2043, 2003). In univariate analyses of the 166 genes that composed our macroarray assay (Table 1), adjusted for age, sex, presence of gadoliniumenhancing MRI lesions, and baseline T2 lesion volume, mean IRs indicated differential responses between the PR and GR groups for 17 genes (P<0.05). Unexpectedly, for all 17 genes, the response, either induction or repression, was greater for patients with a poor response, suggesting an exaggerated IFN-β molecular response in such patients. This hypothesis was confirmed by an analysis of the overall IR frequency in the two groups (FIGS. 8A-B). The figure shows IR frequency for all IRGs for all patients at the first (FIG. 8A) and 6-month (FIG. 8B) IFN-β injection. At the first injection, among the 119 upregulated genes, least-square-mean IRs for the PRs were higher than those for the GRs in 89 genes. Of the 47 repressed genes, IRs in the PRs were lower than in the GRs in 34 genes. Thus, in 123 of 166 genes, an exaggerated response to IFN-β was present in those with a poor response (p<0.001). At the 6-month injection (FIG. 8B), an exaggerated response to IFN-β occurred in 120 of 166 genes (p<0.001).

Using random forest selection, we identified the IRGs most strongly associated with poor or good response status. The random forest technique is a non-parametric ensemble classifier that takes into account the importance of individual variables when selecting each factor (in this case, each IRG), and it is sensitive to the complex interaction and nonlinear dependency between variables. Therefore, we chose to use random forest for variable selection and classification. Table 3 lists the 25 identified genes in which the baseline IR best predicted response status.

TABLE 3
Induction ratios for the 25 interferon-responsive genes
on the custom macroarray that best predicted responder status

| | | Poor | Good | |
|---|---|---|---|---|
| | | Responder | Responder | |
| | Accession | Induction | Induction | |
| Gene Name | Number | Ratio | Ratio | P Value |
| Induced | | | | |
| Genes | | | | |
| TRAIL | U37518 | 6.23 | 4.50 | 0.048 |
| RIG-1 | AF038963 | 5.50 | 4.44 | 0.230 |
| 2-5OAS | NM_002534 | 3.84 | 3.51 | 0.480 |
| STAT1 | M97935 | 3.41 | 3.18 | 0.656 |
| PI3-kinase | NM_006219 | 1.99 | 1.49 | 0.026 |
| IL-15 | U14407 | 1.68 | 1.55 | 0.502 |
| IP-10 | X02530 | 1.55 | 1.33 | 0.109 |
| MMP-1 | M13509 | 1.47 | 1.32 | 0.128 |
| P4HA1 | M24486 | 1.41 | 1.14 | 0.020 |
| caspase 7 | U67319 | 1.37 | 1.13 | 0.040 |
| PDK2 | NM_002611 | 1.31 | 1.02 | 0.047 |
| ATF-2 | X15875 | 1.20 | 1.08 | 0.296 |
| TNF-α | X01394 | 1.13 | 1.01 | 0.283 |
| RGS2 | NM_002923 | 1.11 | 1.05 | 0.603 |
| Repressed | | | | |
| Genes | | | | |
| SNN | NM_003498 | 0.93 | 1.09 | 0.079 |
| hsp90 | X15183 | 0.93 | 1.11 | 0.141 |
| c-myc | L00058 | 0.85 | 0.95 | 0.203 |
| A1-AT | K01396 | 0.84 | 1.04 | 0.199 |
| HLA-DRA | J00194 | 0.78 | 1.01 | 0.074 |
| COMT | M58525 | 0.78 | 0.87 | 0.261 |
| NFκB | M58603 | 0.74 | 0.90 | 0.092 |
| HLA-DP | M83664 | 0.72 | 0.91 | 0.039 |
| TIMP-1 | M59906 | 0.65 | 0.96 | 0.005 |
| CXCR4 | AF005058 | 0.64 | 0.77 | 0.195 |
| IL-2 | NM_000586 | 0.47 | 0.90 | 0.001 |

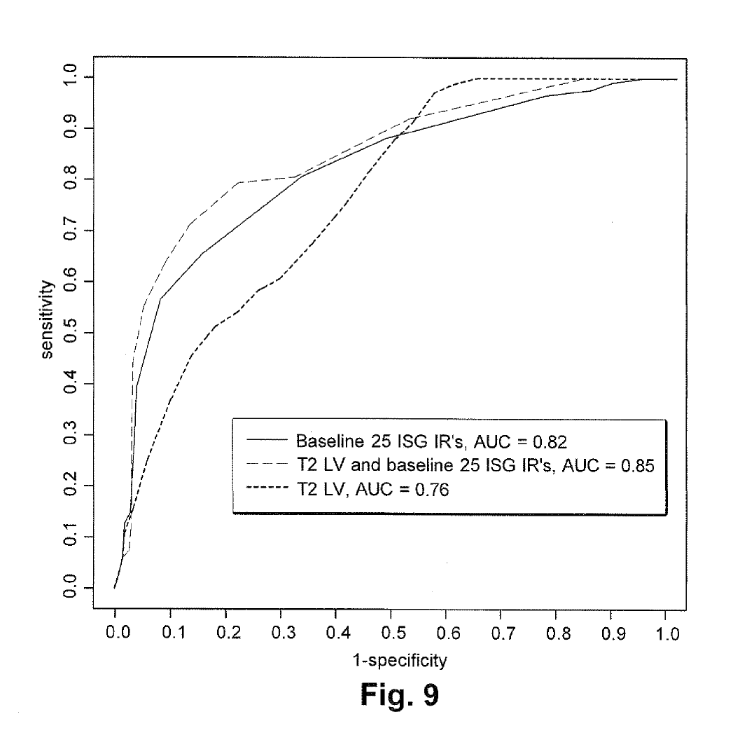

Of the 25 IRGs, 14 were upregulated, and 11 IRGs were repressed in response to the first IFN-β injection. These 25 IRGs were combined in a prediction model, which was used to construct ROC curves to measure its predictive strength (FIG. 9). The predictive strength of the 25-IRG model at the first IFN-β injection was compared with the predictive strength of baseline (pre-IFN-β treatment) T2 lesion volume. A predictive model which combined the baseline T2 lesion volume and the IRs for the 25 IRGs also was constructed. The area under the curve was 0.76 for T2 lesion volume alone, 0.82 for the IRG model, and 0.85 for T2 lesion volume combined with IRGs, indicating that differential IRG induction after the first IFN-β injection was a strong predictor of responder status measured at 6 months using MRI.

The curve shows that the baseline IRG model more strongly predicted the 6-month MRI outcome than did the baseline MRI brain scan.

### Example 2

### Spotting the Macroarray Membranes

Wipe down the entire bench area to be used for spotting to eliminate any excess dust which may interfere with spotting. Next, cover the spotting area with 3 MM paper and set the replicator pins in the Tupperware container of VP110 pin cleaning solution (30 mL of solution to 120 mL of dH₂O). The pins should be about half way submerged in the cleaning solution. While the pins are “soaking” cut enough Hybond-N+ membranes to supply your experiment. For example, while wearing gloves and using a ruler, mark rectangles 74 mm×115 mm on the paper layer used to shield the hybond paper. Make sure not to place too much pressure on the paper and membrane with your hands or elbows and try to have as little contact as possible with the paper covering the center of what will be your membrane. Also, make sure that the membrane doesn't slide around within the paper cover, and use either a clean scalpel and ruler or a clean pair of scissors to cut along the marked lines.

Next, fit each membrane to a nalg-nunc tray by trimming two of the corners and using a pencil to mark a small identifying number on the edge of the membrane. The arch of the paper should be upwards when you place it in the tray so that the edges don't roll up when the membrane is being spotted. If the edges of the membrane need trimmed in order to sit in the tray it is best to trim the bottom as the top will be used for alignment in the phosphor-imager cassette once the experiment is complete.

Dip the pins in the cleaning solution 7-10 times and blot onto VP522 lint free blotting paper allowing them to sit for a count of 5. Dip the pins in dH₂O 7-10 times and again blot and let sit for a count of 5. Repeat this last step with another tub of dH₂O and then dip the pins 7-10 times in isopropanol, blot, and let air dry. Remove the DNA 96 well plates from the −20 C for thawing during this time.

Once the pins are dry and the DNA is completely thawed, place each DNA 96 well plate inside of the correspondingly numbered library copier. Place the pins in the corresponding DNA and do a spot onto the lint free blotting paper in order to “prime” the pins for spotting and place the pins back in the 96 well plate. Place the registration device over top of a tray containing one of the membranes and then remove the pins from the DNA and spot the membrane by gently setting the guide pins into the first hole of the first row of guide holes on the replicator tray. Let the pins sit on the membrane for a count of 5 before removing them back to the DNA plate.

Repeat the previous step for holes 2 and 3 of the first row and then switch to the second tray of DNA and prime its pins. Repeat the previous two steps using holes 1-3 of the second row of guide holes, Switch to the third tray of DNA and prime its pins. Once again, repeat the preceding steps using holes 1-3 of the third row of guide holes. Perform the preceding steps for the rest of the membranes, skipping any priming as that has already been completed. Let all membranes air dry and then store them between two sheets of 3 MM paper until denaturation the following day, and wash the pins again before storage.

### RNA ³²P Labeling for Use in Macroarray Experiments

Add 5 μg of RNA to 10 uL of MILLI Q sterile water (10 μL final volume). Next, add 6 μL T₂₃ACG anchored primer mix (100 pmol/μL) and mix gently but thoroughly. To make T₂₃ACG anchored primer mix (per reaction): primer (3 μL); dNTP (1.5 μL); and dCTP (40 μM) (1.5 μL). For the dNTP, mix equal volumes of 10 mM each dATP, dGTP, and dTTP. Next, incubate for 10 minutes at 72° C. Chill on ice for 2 minutes. Spin down condensation. While incubating, make the following hybridization mix (per reaction): 5× Reverse transcriptase (5 μL); 0.1 M DDT (3 μL); RNAse inhibitor (1 μL); and ³²P dCTP (2 μL).

After 10 minutes are up and the sample has been chilled and spun down, add 11 μL hybridization mix to each reaction and incubate at 42° C. for 2 minutes. Next, add 1.5 μL of Superscript II reverse transcriptase (200 U/μL) to each reaction and mix gently. Incubate for 2 hrs at 42° C. This is a good time to denature the DNA on the macroarray membranes spotted the previous day (e.g., 12-24 hours prior). Pour denaturing buffer (DB) into a large Tupperware container, Place the membranes (DNA side facing up) into the buffer making sure that they are submerged but do not overlap. Leave the membranes in DB for 10 minutes. After 10 minutes, transfer the membranes to a dH₂O bath—with the same care described above. Place the box on the elliptical shaker on low for 10 minutes. Transfer the membranes to ˜600 mL of neutralizing buffer (NB) and place it on the same shaker for 10 minutes more. Transfer the membranes to dH₂O and shake for 10 minutes to rinse the NB from the membranes. Air-dry the membranes on 3 MM paper. Once dry, store the membranes between 2 pieces of 3 MM paper until hybridization.

Once the 2 hours at 42° C. are complete, add 15 mL of 0.1 M sterile filtered NaOH and incubate the tubes at 70° C. for 20 minutes in order to hydrolyze the RNA. After 20 min, add 15 μL of 0.1 M sterile filtered HCl to neutralize the reaction. Prepare the G50 columns by vortexing briefly, breaking off the bottoms, and spinning in the cold room centrifuge or the lab Beckman refrigerated microfuge for 1 minute at 3000 rpm. Carefully place the column into a new Eppendorf tube, so as to not disturb the resin. Slowly add ³²P labeled cDNA directly to the resin (60 μL).

Next, spin the column at 3000 rpm, for 2 minutes in the same centrifuge and remove the column from the Eppendorfs. Flick the flow-through to mix it making sure no samples are pink as this is a sign of incomplete removal of excess isotope. If the sample volumes seem to vary greatly or if Eppendorfs were not changed prior to elution of radioactivity, MICROCON Centrifugal filter devices (MILLIPORE, Billerica, Mass.) can be used to carefully concentrate the samples. This is only necessary in the case of a great difference in volumes (>100 uL difference or as seen fit). Add 1 μL of each tube of flow-through to a corresponding scintillation vial containing 2 mL of scintillation fluid (obtained from the repipette by the radioactive solid waste) (simply add in the whole tip containing the radioactivity). Cap and vortex each scintillation vial to mix.

Use the program 6 slide from under the scintillation reader and run (main menu>automatic counting: select). Check consistency of the scintillation readings, if they are acceptable, then add 50 μL of COT-1 DNA (1 μg/uL) and 5 μL of Poly-A DNA (2 μg/L). Next, prepare the following mixture: 4×SSC (44 μL of 10×SSC-filtered); ddH₂O (45 μL); and 0.1% SDS (1 μL 10% SDS-filtered). Add 90 μL, of mixture to each tube, vortex, spin down drops, incubate in heating block at 95° C. for 5 minutes in order to denature the DNA and hybridize at 65° C. for 2 hours.

This is a good time to prepare membranes. To do so, first dip membrane in water and roll up with DNA on inside of roll. Add to the corresponding pre-warmed hybridization bottle. Add 10 mL of 65° CHURCH buffer and slowly roll the buffer over the membrane so as to avoid getting air bubbles underneath the membrane thereby promoting drying out of the membrane. Place reaction in rotating hybridization oven until hybridization mixture is ready. Add 200 μL of the appropriate hybridization mix to each tube and place immediately back in the hybridization oven and incubate over night.

Prepare 1 L of wash solutions 1 and 3, 2 L of wash solution 2, and pre-warm to 65° C. in a water bath. Once the membranes have hybridized for 16-24 hours remove the bottles two at a time from the hybridization oven, pour off the hybridization mix into a large radioactive waste beaker (this beaker is used only for temporary storage of waste as all waste will be transferred to the 10 L radiation safety issued waste jugs and properly recorded on the waste log sheet), add about 50-100 mLs of wash solution 1, recap the bottle and shake the membrane to rinse it, pour off the rinse and add ¼ to ⅓ of a bottle of wash solution 1 and place the tightly capped bottles back into the hybridization oven and incubate for 15 minutes. After this time is up, discard the buffer, add the same amount of wash solution 2 and incubate for 15 minutes, and repeat the wash step using solution 3.

Once this wash is complete, use shaking to transfer the membrane to the top of the neck of the bottle. Use forceps to remove the membrane, DNA side face up, to a clean Tupperware of dH₂O to rinse off the SDS. Briefly blot the membranes dry on a piece of 3 MM paper and line the membranes up as squarely as possible between two pieces of saran wrap. Using a piece of paper with lines on it as a guide is useful as well as using two pairs of forceps to lay the membranes. Expose the membranes to the Phospholmager cassette about 3 days (see below). Transfer membranes to film cassette and create a hard copy of the data for each set of membranes.

### Capturing Macroarray Data

After the screen of the phosphoimager cassette has been exposed to the membranes for 2-3 days, scan the resulting image using the STORM phosphoimager saving the resulting .gel file to the MACROARRAY folder on Ransoshared. Once the scan is complete and the file is saved, open the file in IMAGEQUANT to capture the data. Begin by checking the preference settings in the “preference” pull down main menu. The “Grid Column Major” should be unchecked; only the “name” and “sum above background” should be selected for the generated volume report under “volume report settings”; and the default background correction should be set to “local median”.

Next, select “Gray scale color adjustment” from the pull down “view” menu. Adjust the color until all of the spots are visible but not over exposed—all spots are still independent from neighboring spots. Select “grid” from the “object” pull-down menu. Enter 24 rows and 36 columns into the window that opens. Draw a grid over one of the membranes making sure one spot is centered per section of the grid. Slight adjustments can be made using the arrow keys or the rotation tool+shift key can be used to rotate the entire grid in the case that the membrane is not nicely aligned.

Once all of the spots are centered, select “background correction” from the “analysis” pull down menu. Select “Local Median” and close the window. Under the “analysis” menu select “Volume Report Settings” and check only “Name” and “Sum above background”. Under “analysis” select “Volume Report”. Select “display” report. Close the window that opens and select yes on the window that appears asking to open the file in Microsoft Excel. Once in Excel, clear the column titles-name and sum above background. Under “File” select “save copy as” and save a copy in the *.cvs (comma delimited) format in the proper sub-folder. Using the arrow keys, shift the grid over the next membrane. Repeat the preceding steps for all remaining membranes. Once all of the data has been captured, save a copy of the *.gel file in the *.tiff format. Open the *.TIFF file in Photoshop Editor and save a *.JPEG of each individual membrane in the *.TIFF file.

From the above description of the invention, those skilled in the art will perceive improvements, changes and modifications. Such improvements, changes, and modifications are within the skill of those in the art and are intended to be covered by the appended claims.

Having described the invention, the following is claimed:

1. A method of determining the efficacy of interferon-beta (IFN-β) therapy in a subject with multiple sclerosis (MS), the method comprising the steps of:
obtaining a biological sample from the subject; and
determining the expression level of at least one interferon-regulated gene (IRG) and/or variant thereof;
wherein increased or decreased expression of the at least one IRG and/or variant thereof as compared to a control indicates that the subject will respond poorly to IFN-β therapy.

2. The method of claim 1, the biological sample comprising whole blood.

3. The method of claim 2, further comprising isolating RNA from the whole blood sample.

4. The method of claim 1, further including administering a dose of IFN-β to the subject prior to obtaining the biological sample.

5. The method of claim 4, further including obtaining the biological sample in less than about 12 hours after administration of the IFN-β dose.

6. A method for screening an agent that can be used to treat MS, the method comprising the steps of:
providing a population of peripheral blood mononuclear cells (PBMCs) from a subject with MS that is a poor responder to IFN-β therapy;
administering an agent to the PBMCs; and
determining the expression level of at least one IRG and/or variant thereof in one or more of the PBMCs.

7. The method of claim 6, wherein increased or decreased expression of the at least one IRG and/or variant thereof as compared to a control indicates that the agent is not a candidate for MS therapy.

8. A method of treating a subject with MS, the method comprising the steps of:
obtaining a biological sample from the subject;
determining the expression level of at least one IRG and/or variant thereof; and
administering to the subject a therapeutically effective amount of at least one agent, besides IFN-β, if expression of one or more of the at one IRG and/or variant thereof is increased or decreased as compared to a control.

9. The method of claim 8, the biological sample comprising whole blood.

10. The method of claim 9, further comprising isolating RNA from the whole blood sample.

11. The method of claim 8, further including administering a dose of IFN-β to the subject prior to obtaining the biological sample.

12. The method of claim 11, further including obtaining the biological sample in less than about 12 hours after administration of the IFN-β dose.

13. A method of treating a subject with MS, the method comprising the steps of
obtaining a biological sample from the subject;
determining the expression level of at least one IRG and/or variant thereof; and
administering to the subject a therapeutically effective amount of natalizumab if expression of the at least one IRG and/or variant thereof is increased or decreased as compared to a control.

14. The method of claim 13, the biological sample comprising whole blood.

15. The method of claim 14, further comprising isolating RNA from the whole blood sample.
